(19) United States
(12) Patent Application Publication
(10) Pub. No.: US 2026/0209172 A1
(43) Pub. Date: Jul. 23, 2026

(54) TUNABLE AND LONG-LIVED LIGHT REFLECTION FROM LIQUID CRYSTALS USING HYDRAZONE DOPANTS

(71) Applicant: Trustees of Dartmouth College, Hanover, NH (US)

(72) Inventors: Ivan Abrahamian, Hanover, NH (US); Indu Bala, Hanover, NH (US)

(73) Assignee: Trustees of Dartmouth College, Hanover, NH (US)

(21) Appl. No.: 19/139,458

(22) PCT Filed: Dec. 13, 2023
(86) PCT No.: PCT/US2023/083908

§ 371 Date: Jun. 16, 2025

Related U.S. Application Data
(60) Provisional application No. 63/432,582, filed on Dec. 14, 2022.

(51) Int. Cl. C07C 251/86; C07C 245/08; C09K 19/58; G02F 1/01

(52) U.S. Cl. CPC C07C 251/86; C07C 245/08; C09K 19/586; G02F 1/0126; C07C 2603/90

(57) ABSTRACT

Embodiments of the present disclosure pertain to at least one chiral compound with a photo-switchable unit. Additional embodiments of the present disclosure pertain to substrates that include one or more compounds of the present disclosure. Further embodiments of the present disclosure pertain to methods of changing a color of one or more regions of a substrate of the present disclosure by applying light to the one or more regions of the substrate.

### CROSS-REFERENCE TO RELATED APPLICATIONS

This application claims priority to U.S. Provisional Patent Application No. 63/432,582, filed on Dec. 14, 2022. The entirety of the aforementioned application is incorporated herein by reference.

### STATEMENT REGARDING FEDERALLY SPONSORED RESEARCH

This invention was made with government support under 2104464 awarded by the National Science Foundation. The government has certain rights in the invention.

### BACKGROUND

Existing multi-chromic systems have numerous limitations, including an inability to lock-in a certain color, and a limited color reflection timeframe. Numerous embodiments of the present disclosure aim to address the aforementioned limitations.

### SUMMARY

In some embodiments, the present disclosure pertains to at least one chiral compound. In some embodiments, the compound includes, without limitation:

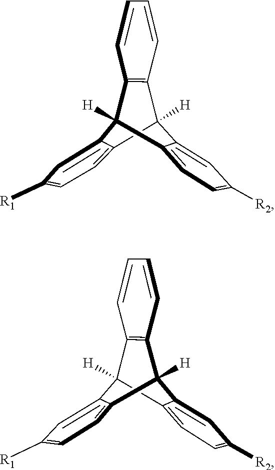

derivatives thereof, or combinations thereof. In some embodiments, at least one of R₁ and R₂ includes a photo-switchable unit.

Additional embodiments of the present disclosure pertain to substrates that include one or more compounds of the present disclosure. In some embodiments, the substrate includes one or more regions. In some embodiments, the one or more regions are associated with one or more compounds of the present disclosure. In some embodiments, the substrate includes a plurality of different regions. In some embodiments, the different regions of the substrate include different compounds of the present disclosure. In some embodiments, the different regions of the substrate include the same compounds of the present disclosure.

Additional embodiments of the present disclosure pertain to methods of changing a color of one or more regions of a substrate of the present disclosure by applying light to the one or more regions of the substrate. In some embodiments, the applying of the light to the one or more regions of the substrate includes: (1) applying a first light to one or more regions of the substrate such that the first light changes the color of the one or more regions of the substrate to a first color, and (2) applying a second light to one or more regions of the substrate such that the application of the second light changes the color of the one or more regions of the substrate to a second color. In some embodiments, the aforementioned light application steps may be repeated multiple times to form a color pattern on a substrate.

### FIGURES

FIG. 1 illustrates a method of changing a color of one or more regions of a substrate in accordance with various embodiments of the present disclosure.

FIGS. 2A-2E illustrate the synthesis, use and characterization of various chiral dopants. FIG. 2A illustrates the synthesis of the triptycene based photochromic chiral dopants (R, R)-1/(S, S)-1 to (S, S)-7 from the corresponding enantiomers (R, R)-8 and (S, S)-8, respectively. FIG. 2B shows a circular dichroism (CD) spectrum of the triptycene diamines (top) and triptycene-based hydrazone photo-switches (bottom). FIG. 2C shows the photoisomerization process of the triptycene-hydrazone chiral dopants. The switch toggles mainly between the ZZ isomer to predominantly the EE upon 442 and 340 nm light irradiation, respectively. FIG. 2D shows spiral textures corresponding to (R, R)-1 (right), and (S, S)-1 (left) doped 5CB. FIG. 2E shows UV-vis spectra of the different PSSs that can be obtained as a function of applied wavelength. The ratio of the isomers (ZZ, EE and ZE) obtained at each PSS is also indicated.

FIGS. 3A-3C summarize an application of the (S, S)-1 chiral dopant in changing substrate color. FIG. 3A shows polarized optical micrographs (POMs) of the (S, S)-1 sample (0.45% in κCB) in Cano wedge cells after irradiations with 442, 410, 394, 375, 365 and 340 nm light. FIG. 3B shows the change in the transmittance spectra of 5CB doped with (S, S)-1 as a function of irradiation wavelengths. FIG. 3C shows photomicrographs of red, green and blue (RGB) colored heart-shaped images obtained by irradiating 5 μm thick planar cells (with parallel rubbing) of a masked CLC mixture (3.1 mol % doped (S, S)-1 in 5CB).

FIGS. 4A-4G summarize additional applications of the (S, S)-1 chiral dopant in changing substrate color. FIG. 4A shows the change in the transmittance spectra of 5CB doped with (S, S)-1 as a function of different irradiation times with 442 nm wavelength of light. FIG. 4B shows a schematic of DLP patterning setup used to create multicolor images by controlling irradiation time on a particular area. FIG. 4C shows an image of LC cell mounted and sample patterns on a clear microscope slide. FIG. 4D shows patterns projected and produced for DLP patterning experiments of Natalia Goncharova's “Cyclist” with foreground pattern (left), highlight pattern (middle), and multicolor image produced on LC cell (right). FIGS. 4E-4F show Edvard Munch's “The Scream” (FIG. 4E), “Checkerboard” (FIG. 4F) and Van Gogh's “Starry Night” (FIG. 4G) images produced on the LC cell. Transmittance spectra and all the DLP patterned multicolored images shown here are recorded in 5 μm thick planar cells (with parallel rubbing) filled with CLC mixture (3.2 mol % doped (S, S)-1 in 5CB).

FIG. 5 summarizes additional experimental results related to the locking-in of red, blue and green colors on a substrate that includes a compound of the present disclosure (i.e., (S,S)-1).

FIG. 6 summarizes additional experimental results related to the locking-in of red, blue and green colors on a substrate that includes a compound of the present disclosure (i.e., (S,S)-Azo).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Materials that exhibit multichromic responses as a function of light are highly desirable. Such systems have applications ranging from smart surfaces, display screens, and anti-counterfeit devices.

For instance, the photo modulation of the helical pitch of cholesteric liquid crystals (CLCs) results in dynamic and colored canvases that can potentially be used in applications ranging from energy-efficient displays to color filters, anti-counterfeiting tags and LC lasers. However, the challenge in attaining these functions is the development of photo switchable chiral dopants with optimal properties that can modulate the CLCs in a controllable and multistage manner, and to afford long-lived multi-color reflections from the LC surface.

Moreover, existing multi-chromic systems have numerous limitations, including an inability to lock-in a certain color, and a limited color reflection timeframe. Numerous embodiments of the present disclosure aim to address the aforementioned limitations.

### Compounds

In some embodiments, the present disclosure pertains to at least one chiral compound. In some embodiments, the compound includes, without limitation:

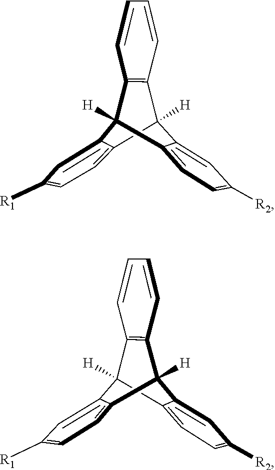

derivatives thereof, or combinations thereof.

In some embodiments, the compound includes the following structure or a derivative thereof:

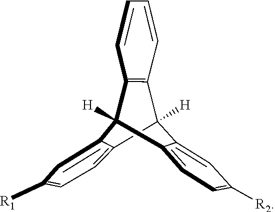

In some embodiments, the compound includes the following structure or a derivative thereof:

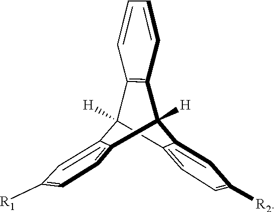

In some embodiments, at least one of R₁ and R₂ includes a photo-switchable unit. In some embodiments, R₁ and R₂ each independently includes, without limitation, alkyl groups, alkene groups, alkenyl groups, alkoxy groups, hydroxy groups, aryl groups, ketone groups, aldehyde groups, amine groups, amide groups, imine groups, carboxyl groups, carboxylic acid groups, ester groups, thiol groups, sulfoxide groups, alcohol groups, alkyne groups, azide groups, hydrazide groups, hydrazone groups, azo groups, cyano groups, nitro groups, halogens, a methyl group, a polymer, a substrate component, a photo-switchable unit, or combinations thereof.

In some embodiments, R₁ includes a photo-switchable unit. In some embodiments, R₂ includes a photo-switchable unit. In some embodiments, R₁ and R₂ each include a photo-switchable unit. In some embodiments, R₁ and R₂ include the same photo-switchable unit. In some embodiments, R₁ and R₂ include different photo-switchable units.

The compounds of the present disclosure may include various types of photo-switchable units. For instance, in some embodiments, the photo-switchable unit is bistable. In some embodiments, the photo-switchable unit includes, without limitation, a hydrazone unit, an azobenzene unit, or combinations thereof. In some embodiments, the photo-switchable unit includes a hydrazone unit. In some embodiments, the photo-switchable unit includes an azobenzene unit.

In some embodiments, the photo-switchable unit includes, without limitation:

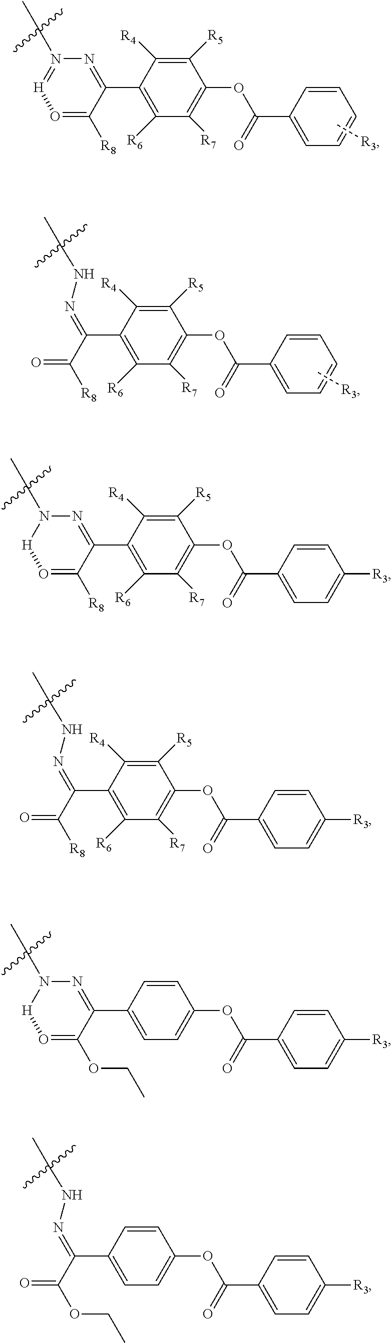

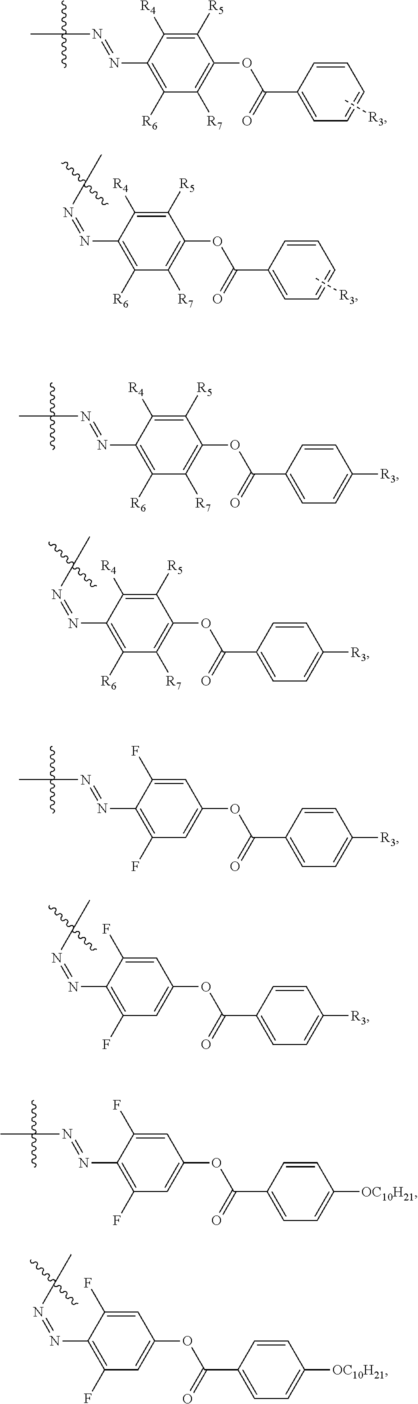

derivatives thereof, or combinations thereof.

In some embodiments, R₃, R₄, R₅, R₆, R₇ and R₈ each independently includes, without limitation, H, alkyl groups, alkene groups, alkenyl groups, alkoxy groups, hydroxy groups, aryl groups, ketone groups, aldehyde groups, amine groups, amide groups, carboxyl groups, carboxylic acid groups, ester groups, thiol groups, sulfoxide groups, alcohol groups, alkyne groups, azide groups, halogens, CN, OC₁₀H₂₁, —OCH₃, NO₂, F, N(CH₃)₂, a methyl group, a polymer, a substrate component, a photo-switchable unit, or combinations thereof.

In some embodiments, each of R₁ and R₂ in the compounds of the present disclosure includes the following structure:

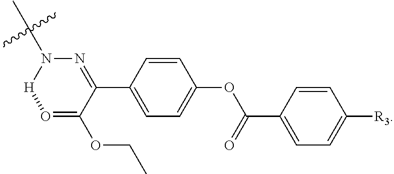

In some embodiments, R₃ is OC₁₀H₂₁.

In some embodiments, each of R₁ and R₂ in the compounds of the present disclosure includes the following structure:

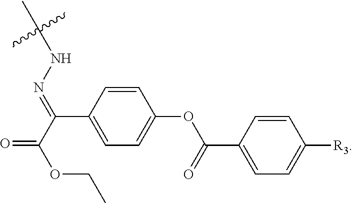

In some embodiments, R₃ is OC₁₀H₂₁.

The compounds of the present disclosure may include various derivatives. In some embodiments, the derivative compounds include one or more moieties derivatized with one or more functional groups. In some embodiments, the one or more functional groups include, without limitation, alkyl groups, alkoxy groups, methyl groups, methoxy groups, amine groups, nitro groups, cyano groups, acyl groups, halogens, chlorine, fluorine, bromine, iodine, deuterium, alkanes, alkenes, ethers, alkynes, alkoxyls, aldehydes, carboxyls, hydroxyls, hydrogens, sulfurs, linkers, hydrogen groups, tracing agents, or combinations thereof.

### Substrates

The compounds of the present disclosure can have various configurations. For instance, in some embodiments, the compounds of the present disclosure are associated with a substrate. In some embodiments, the compounds of the present disclosure are embedded with the substrate. In some embodiments, the compounds of the present disclosure are embedded with the substrate through one or more of the R₁, R₂, R₃, R₄, R₅, R₆, R₇, or R₈ groups. In some embodiments, the substrate includes, without limitation, a film, a liquid crystal film, a display panel film, a mask surface, a polymer, a polydimethylsiloxane film, a display screen, a paper, or combinations thereof. In some embodiments, the substrate includes a liquid crystal film.

Additional embodiments of the present disclosure pertain to substrates that include one or more compounds of the present disclosure. In some embodiments, the substrate includes one or more regions. In some embodiments, the one or more regions are associated with one or more compounds of the present disclosure. In some embodiments, the substrate includes a plurality of different regions. In some embodiments, the different regions of the substrate include different compounds of the present disclosure. In some embodiments, the different regions of the substrate include the same compounds of the present disclosure.

### Methods of Changing a Substrate Color

Additional embodiments of the present disclosure pertain to methods of changing a color of one or more regions of a substrate by applying light to the one or more regions of the substrate. The one or more regions of the substrate include one or more compounds of the present disclosure, which facilitate the change in color.

Various types of light may be applied to the substrates of the present disclosure. For instance, in some embodiments, the light includes a wavelength ranging from about 300 nm to about 2500 nm. In some embodiments, the light includes a wavelength ranging from about 500 nm to about 2500 nm. In some embodiments, the light includes a wavelength ranging from about 300 nm to about 900 nm. In some embodiments the light includes a wavelength of about 450 nm to about 800 nm. In some embodiments the light includes a wavelength of about 340 nm to about 450 nm. In some embodiments, the application of different wavelengths of light will have different color change effects on the one or more regions of the substrate.

In some embodiments, the stimulus includes light from a digital light projector. In some embodiments, the digital light projector is operable to provide native projection optics for macroscale patterning, microscopic optics for microscopic resolution patterning, or combinations thereof.

Light may be applied to one or more regions of the substrates of the present disclosure for various periods of time. For instance, in some embodiments, light is applied to one or more regions of a substrate for a time period ranging from about 1 second to about 180 seconds. In some embodiments, light is applied to one or more regions of a substrate for a time period ranging from about 1 second to about 120 seconds. In some embodiments, light is applied to one or more regions of a substrate for a time period ranging from about 1 second to about 60 seconds. In some embodiments, light is applied to one or more regions of a substrate for a time period ranging from about 1 second to about 20 seconds. In some embodiments, light is applied to one or more regions of a substrate for a time period ranging from about 4 seconds to about 16 seconds. In some embodiments, the application of light for different periods of time will have different color change effects on the one or more regions of the substrate.

Without being bound by theory, the change in color of a substrate may occur through various mechanisms. For instance, in some embodiments, the change in color occurs by switching of the photo-switchable unit of the one or more compounds of the present disclosure from a first configuration to a second configuration. In some embodiments, the second configuration has different light absorption properties than the first configuration. In some embodiments, the light converts the configuration of a compound's photo-switchable unit from a Z-state to an E-state. In some embodiments, the light converts the configuration of a compound's photo-switchable unit from an E-state to a Z-state.

The application of light may have various effects on a substrate. For instance, in some embodiments, the application of the light forms a color pattern on the substrate. In some embodiments, the application of light forms a multi-color pattern on the substrate. Additionally, as set forth in more detail here, light may be applied to one or more regions of a substrate in different manners to achieve different patterns on the substrate.

For instance, in some embodiments, the same wavelength of light is applied to different regions of the substrate to result in the same color change in the different regions of the substrate. In some embodiments, different wavelengths of light are applied to different regions of the substrate to result in different color changes in the different regions of the substrate. In some embodiments, different wavelengths of light are applied to the same regions of the substrate to result in different color changes in the same regions of the substrate.

In some embodiments, light is applied to the same region of the substrate for the same period of time to result in the same color change in the same region of the substrate. In some embodiments, light is applied to the same region of the substrate for different periods of time to result in different color changes in the same region of the substrate. In some embodiments, light is applied to different regions of the substrate for the same periods of time to result in the same color change in the different regions of the substrate. In some embodiments, light is applied to different regions of the substrate for different periods of time to result in different color changes in the different regions of the substrate.

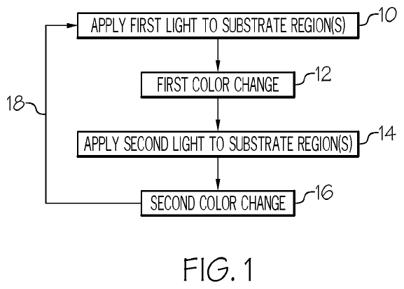

In some embodiments illustrated in FIG. 1, the applying of the light to the one or more regions of the substrate includes: (1) applying a first light to one or more regions of the substrate (step 10) such that the first light changes the color of the one or more regions of the substrate to a first color (step 12); and (2) applying a second light to one or more regions of the substrate (step 14) such that the application of the second light changes the color of the one or more regions of the substrate to a second color (step 16). In some embodiments, the aforementioned light application steps may be repeated multiple times to form a color pattern on a substrate (step 18).

Additionally, the methods illustrated in FIG. 1 can have numerous embodiments. For instance, in some embodiments, the first light and the second light have different wavelengths. In some embodiments, the first light and the second light have the same wavelengths. In some embodiments, the first light and the second light are applied to one or more regions of the substrate for different periods of time. In some embodiments, the first light and the second light are applied to one or more regions of the substrate for the same period of time. In some embodiments, the first light and the second light are applied to the same region of the substrate. In some embodiments, the first light and the second light are applied to different regions of the substrate.

In some embodiments, the application of light to one or more regions of a substrate includes: (1) applying a first light to one or more regions of the substrate such that the first light changes the color of the one or more regions of the substrate; (2) applying a second light to the one or more regions of the substrate after the application of the first light such that the second light reverses the change in the color of the one or more regions of the substrate. In some embodiments, the first light includes light at a first wavelength. In some embodiments, the second light includes light at a second wavelength. In some embodiments, the first wavelength is about 442 nm. In some embodiments, the second wavelength is about 340 nm.

The methods of the present disclosure may facilitate different color changes on a substrate. For instance, in some embodiments, the change in color includes a change in color to red, green, blue, purple, pink, orange, yellow, or combinations thereof. In some embodiments, the change in color includes a change in color to red, green, blue, or combinations thereof.

In some embodiments, the change in color is in the form of a pattern on the substrate. In some embodiments, the change in color is stable. In some embodiments, the change in color is reversible. For instance, in some embodiments, the change in color is reversible upon the application of light to the substrate.

The methods of the present disclosure may apply light to various types of substrates. Suitable substrates were described previously. For instance, in some embodiments, the substrate includes a plurality of different regions. In some embodiments, each of the plurality of different regions are associated with the one or more compounds of the present disclosure. In some embodiments, the different regions of the substrate include different compounds of the present disclosure. In some embodiments, the different compounds facilitate different changes of color upon the application of light. In some embodiments, the different compounds facilitate same changes of color upon the application of light.

In some embodiments, different regions of a substrate include the same compounds of the present disclosure. In some embodiments, the same compounds facilitate the same changes of color upon the application of light. In some embodiments, the same compounds facilitate different changes of color upon the application of different wavelengths of light.

In some embodiments, the methods of the present disclosure also include a step of applying the one or more compounds of the present disclosure to one or more regions of a substrate. Application of compounds to a substrate can occur by various methods. For instance, in some embodiments, the applying occurs by a method that includes, without limitation, spraying, doping, embedding, in situ formation, or combinations thereof.

The methods of the present disclosure can have various advantageous applications. For instance, in some embodiments, the methods of the present disclosure may be utilized for authentication, counterfeit monitoring, pattern formation, display of information, or combinations thereof.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Multistage and Multicolor Liquid Crystal Reflections Using a Chiral Triptycene Photo Switchable Dopant

In this Example, Applicant reports on the structure property analysis of a series of photo switchable chiral dopants that combine the large geometrical change and bi-stability of hydrazone switches with the efficient helical pitch induction of the new chiral motif, triptycene. These studies have elucidated the effects that conformational flexibility, dispersion forces, and π-π interactions have on the chirality transfer ability of the dopant. Applicant also used a dopant in designing LC surfaces whose reflected wavelength (450-800 nm) can be controlled and tuned as a function of irradiation wavelength or time dependent isomer ratio.

The bi-stability of the hydrazone switch allowed Applicant to lock-in different reflected colors from the surface, including the primary colors red, green, and blue, for extended periods of time. Finally, irradiation time with visible light (442 nm) combined with a simple digital light processing microscope projection setup was used in drawing numerous multi-colored images on an LC canvas, showcasing the fine control this dopant yields over the LC assembly. This Example demonstrates how the combination of a bistable photo switch with a chiral motif having a strong helical twisting power can result in adaptive LC surfaces whose properties can be tuned with exceptional efficiency.

### Example 1.1. Background

The noninvasive and remote control over the properties of self-organized supramolecular architectures is a preferred step towards the fabrication of smart molecular materials and devices. One approach to this end is to integrate molecular photo switches into soft supramolecular materials as a means to reversibly manipulate their dynamic superstructures with the high spatiotemporal resolution of light. A notable illustration of this concept is the use of photo switchable chiral dopants in controlling the supramolecular helical self-assembly of liquid crystals (LCs). In such LC materials (known as cholesteric LCs (CLCs)) the periodic pitch length, P, controls the assembly's ability to selectively reflect light. Adjusting the pitch of such assemblies (and hence reflected wavelength/color) and locking them in at desired value(s) using photo switchable dopants is one of the main challenges in the field.

Azobenzene photo switches, in conjunction with binaphthyl chiral cores have been extensively used as photo switchable chiral dopants that allow for the light-controlled manipulation of the reflected colors from CLCs. However, and in general, the relative fast thermal isomerization of the Z isomer of azobenzenes impedes the locking-in of the supramolecular helices, and hence results in transient photophysical properties.

While bistable diarylethene (DA) switches have been used to address the aforementioned issues, resulting in limited cases where the reflected colors can be stable for days, this approach usually suffers from several drawbacks. In general, and because of the limitations with the shape change upon photoisomerization, DA switches result in low helical twisting powers (β, i.e., their ability to induce twisting in an achiral nematic LC) and/or change in β (Δβ), thus requiring large amounts of doping and/or restricting the wavelength reflection range. These constraints also preclude the use of such dopants in designing LC surfaces that can reflect multiple colors simultaneously, which is a limiting factor in the potential applicability of such systems. Moreover, the DA dopants usually result in multiple helical domains and orientation disorder, which decreases the optical efficiency of the LC system.

To address the latter issues, intrinsically chiral DA photo switch dopants were recently developed, which showed enhanced switching and light-reflection properties including multistage color tuning. Nonetheless, and although the device itself was reusable after half a year, the obtained figures (i.e., reflected colors) were reported to be stable for only up to 4 hours. Finally, there are no general guiding principles nor hypotheses that can be used in designing chiral dopants that have large β and Δβ values, and hence, practitioners rely on trial and error in their efforts. Addressing this obstacle can open the door for designing efficient chiral dopants that can be used in the myriad of applications that rely on high performance adaptive LCs.

Photochromic hydrazones, which are characterized by their bistability and significant geometric shape-change upon photoisomerization, are primed to address this obstacle in the field. However, Applicant's first attempt in modulating LC properties using a hydrazone based switchable dopant with isosorbide being the chiral motif, resulted in low β values (35 and 57 μm⁻¹ for the Z-rich E-rich states, respectively). The latter limited the obtained reflectance wavelengths to the near infrared (NIR) range. Nevertheless, the photoisomerization resulted in a multi-stage change in the reflection wavelength and triggered a rare isothermal phase transition from cholesteric to chiral smectic phase, enabling the creation of a light-gated optical window. To address the issue of low β and Δβ values, which is in general a limitation of isosorbides, and very importantly to extend the limited structural space of chiral motifs available to practitioners in the field, Applicant decided to develop triptycene as the chiral core of the switchable dopant.

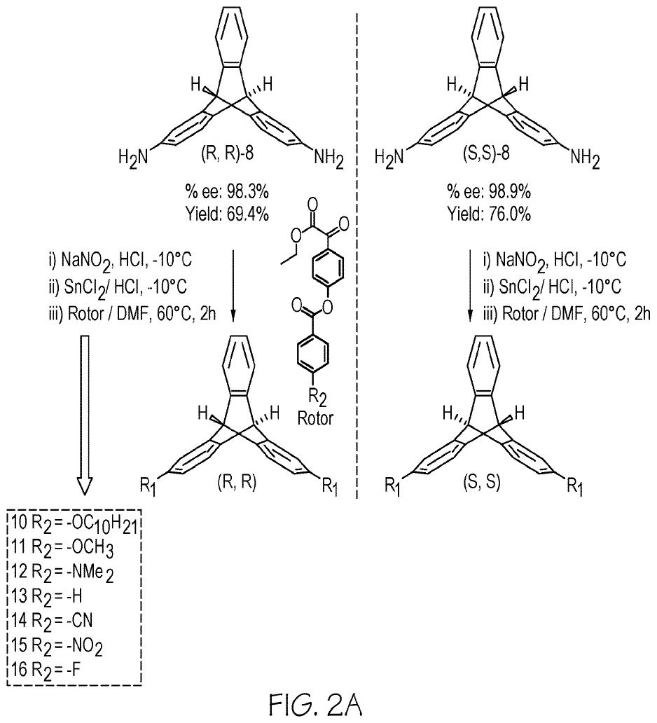

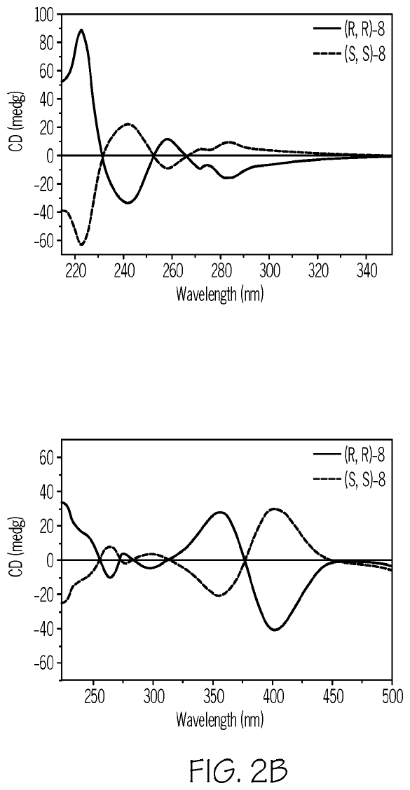

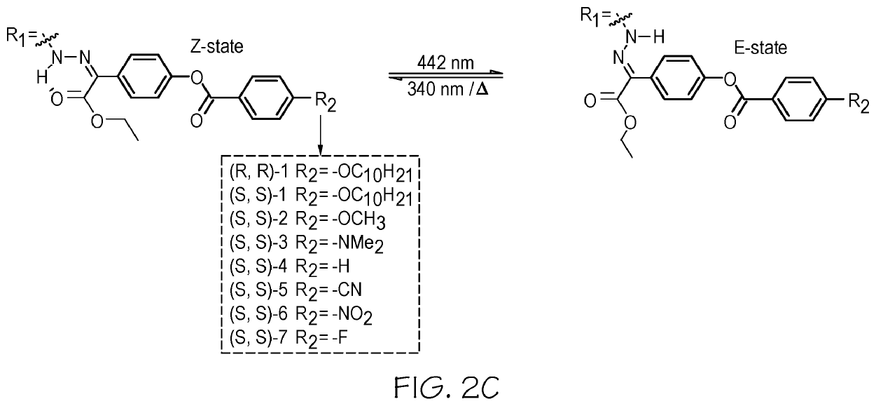

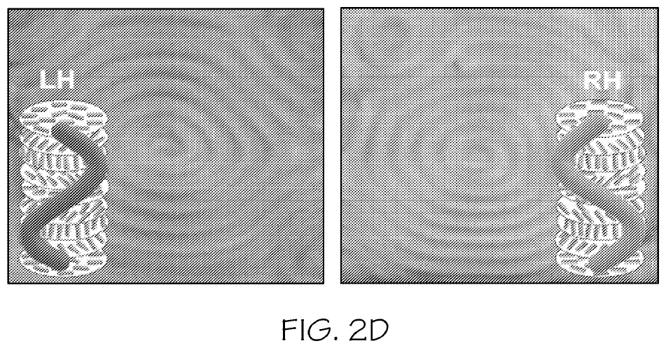

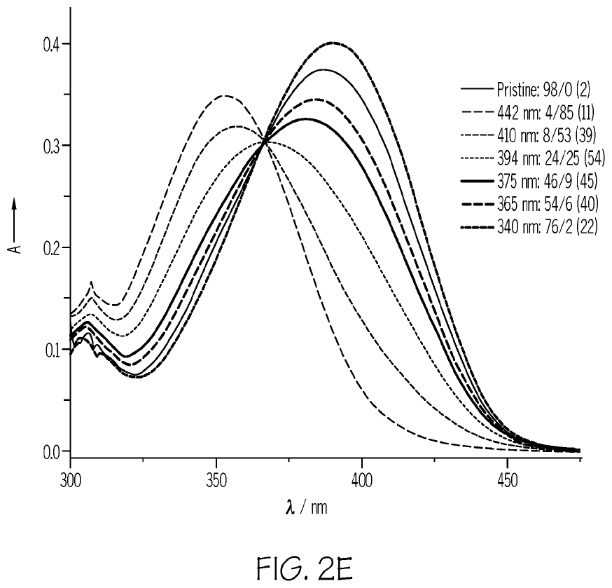

In this Example, Applicant demonstrates for the first time how triptycene can be used as the chiral building block of photo switchable dopants (FIG. 2A), and how its combination with bistable hydrazones results in tunable, multi-stage, and long-lived multi-color reflections in CLCs. Applicant first studied the effect of the pendent group (R₂ in FIG. 2A) on the β and Δβ values of the dopant. These studies showed that conformational flexibility, dispersion forces and π-π interactions play an important role in determining these values.

Next, Applicant used the relatively large β (53 μm⁻¹ for ZZ-rich state and 107 μm⁻¹ for EE-rich state) and Δβ (change in β upon photoisomerization) values (54 μm⁻¹) of the preferred dopant (S, S)-1, in combination with the unique properties that bi-stability affords (i.e., locking-in of different isomer ratios of the hydrazone, and hence, different properties in the same material), to access various non-equilibrated persistent states (i.e., kinetically trapped ones) of the LC self-assembly, as a function of wavelength or irradiation time, resulting in surfaces whose reflection of the primary red, green, and blue (RGB) colors, can be indefinitely locked-in. These properties, in combination with an easy to implement digital light processing (DLP) microscope projection setup, enabled Applicant to develop reconfigurable and rewritable canvases that display photo-reversible and stable multi-color images. These photo responsive multi-color reflective surfaces require no power to maintain their properties and are a promising strategy for the advancement of long-lived, light-driven and configurable energy efficient displays, among other applications.

### Example 1.2. Results and Discussions

To synthesize the chiral dopants (R, R)-1/(S, S)-1-(S, S)-7 (FIG. 2C), Applicant first prepared the enantiomerically pure diamines (R, R)-8 and (S, S)-8. To this end, Applicant first synthesized racemic 2,6-diaminotriptycene (rac-8) by nitrating triptycene followed by reduction using a reported procedure. The racemate was then resolved into its enantiomers (using a CHIRALCEL OD-H column (MeOH+0.1% Et₂NH) yielding an enantiomeric excess (% ee) of 98.3 and 98.9% for (R, R)-8 and (S, S)-8, respectively.

The enantiomers were characterized by circular dichroism (CD) spectroscopy, which yielded the expected mirror images (FIG. 2B, top). The absolute configuration of the enantiomers was assigned by relying on a previous report. With the enantiomers in hand, the target dopants were synthesized in a straightforward manner (FIG. 2A) and characterized by using NMR and high resolution mass spectrometry (HRMS). The CD spectra for the enantiomeric pair (R, R)-1 and (S, S)-1 showed excellent mirror images indicating no loss in chirality during the reaction cycle (FIG. 2B, bottom). The reason for directly attaching the photo switchable units to the chiral motif was to amplify the geometrical change upon photoisomerization, while the LC compatible 4-decyloxyphenyl benzoate units in dopant 1 were used to enhance the dopant's solubility in the LC matrix. Applicant's hypothesis was that these structural elements will result in high β and Δβ values. To further test this hypothesis and investigate the effect of the terminal decyl alkoxy chain (—OC₁₀H₂₁) on the dopant's properties, Applicant replaced this unit with —OMe, —NMe₂, —H, —CN, —NO₂ and —F groups. The S configuration was chosen for these studies because (S, S)-1 exhibited a higher Δβ value than (R, R)-1.

The photophysical and photoisomerization (FIG. 2C) properties of the enantiomeric dopants (1-7) were studied using UV-vis and ¹H nuclear magnetic resonance (NMR). For instance, irradiation of pristine samples of (S, S)-1 (λₘₐₓ=390 nm, ε=40120 M⁻¹ cm⁻¹, 98% ZZ) in toluene with 442 nm light affords a photostationary state (PSS₄₄₂) consisting of 85% EE (λₘₐₓ=354 nm, ε=36546 M⁻¹ cm⁻¹) and a minor ZE population (11%) along with minimal amount of the ZZ isomer (4%) (FIG. 2E). Upon irradiation with 340 nm light, the majority ZZ state (76%) is restored along with minor fraction of the ZE (22%) and negligible amount of EE state (2%). The quantum yields for the Z→E isomerization and its reverse process were measured to be 1.79±0.05 and 3.26±0.36%, respectively, while the thermal isomerization half-life of the photo switch was calculated to be 11 years.

The fatigue resistance of the switch was also studied, and minimal change in absorption intensity was observed after 10 consecutive switching cycles. Applicant also took advantage of the bi-stability of the system to obtain and lock-in different 7E isomer ratios (i.e., different PSSs) of (S, S)-1 as a function of irradiation wavelength (FIG. 2E). The photophysical and photoisomerization studies were also performed for the other enantiomer (R, R)-1 as well as the other enantiomeric dopants 2-7. These switches did not exhibit discernible substituent effects on the absorption wavelengths, photostationary states, quantum yields or half-lives, relative to (S, S)-1.

TABLE 1
Helical twisting powers, β (μm⁻¹) in 5CB^{a}.

| Chiral | ZZ-rich state, | EE-rich state, | |
|---|---|---|---|
| dopant | PSS, 340 nm | PSS, 442 nm | Δβ^{b} |
| (R, R)-1 | −59 | −109 | 50 |
| (S, S)-1 | +53 | +107 | 54 |
| (S, S)-2 | +52 | +85 | 33 |
| (S, S)-3 | +49 | +87 | 38 |
| (S, S)-4 | +28 | +76 | 48 |
| (S, S)-5 | +47 | +85 | 38 |
| (S, S)-6 | +31 | +55 | 24 |
| (S, S)-7 | +35 | +59 | 24 |

^{a}β values were calculated using Grandjean-Cano wedge method with EHC Co. Ltd. KCRK07 wedge cell at room temperature (~22° C.).
^{b}Difference in helical twisting power (Δβ) between the PSS, 340 nm (majority ZZ-isomer) and PSS, 442 nm (majority EE-isomer).

To study the effect of the triptycene based photochromic dopants on the LC properties of achiral nematic hosts, Applicant doped them into the 5CB LC host resulting in cholesteric phases (FIG. 2D). The dopants showed optimal solubility in the nematic host and induced opposite handedness when doped with enantiomeric pair (R, R)-1 and (S, S)-1.

The β values of the dopants were measured using the Grandjean-Cano wedge method. For example, (R, R)-1 in 5CB has a β value of −59 μm⁻¹ at PSS₃₄₀ (ZZ rich state) which changes to −109 μm⁻¹ at PSS₄₄₂ (EE rich state), yielding a relatively large Δβ value of 50 μm⁻¹. Similarly, the β values were obtained for (S, S)-1 in 5CB as well (+53 μm⁻¹ at PSS₃₄₀, +107 μm⁻¹ at PSS₄₄₂ and Δβ of 54 μm⁻¹. The slight difference in the β values is likely attributed to the subtle variations in their enantiopurity. As can be noted from Table 1, the E isomer of these and subsequent dopants have higher β values (i.e., better interactions with the LC host) than the Z isomer. Without being bound by theory, Applicant attribute this observation to the conformational flexibility of the E isomer, which allows one to adopt a more favorable structure that can interact better with the host LC, vs the more rigid Z isomer, whose hydrazone core is locked in place because of the intramolecular H-bond.

Exchanging the long decyloxy chain in 1 to a shorter methoxy chain in 2 does not change the β value of the Z isomer, whereas the value decreases by 20 μm⁻¹ for the E isomer. Without being bound by theory, Applicant hypothesizes that this change results from the loss of the dispersion interactions between the alkoxy tail and the alkyl chain of 5CB. The β values of 2 (—OMe) are similar to 3 (—NMe₂), indicating that the electron donating capability of the substituent is not playing a major role in determining these values. Nonetheless, comparing these two dopants with 6 (—NO₂) and 7 (—F) it becomes evident that π-π interaction between the dopant and LC host are important as well. These interactions are stronger in both the E and Z forms of 2 and 3 because they have an electron rich aromatic core that interacts better with the electron poor n-core of 5CB. Whereas the electron poor cores of 6 and 7 results in weaker interactions.

These results also showcase that highly polarizable groups such as —F, which result induce strong dipole moments, do not always yield large β values. The exception in the electron withdrawing series is dopant 5, which has a —CN group making its n-core similar to that of 5CB, allowing it to interact with the LC host as good as 2 and 3 do. That is, the structural similarity is enhancing the interactions between host and guest. Finally, the β value of the unsubstituted dopant 4 in the Z form is similar to that of 6 and 7, indicating that these three dopants have adopted the minimal interaction possible in this configuration. On the other hand, the β value in the E form is intermediate between the electron donating and pushing groups, clearly showcasing the importance of conformational flexibility of the dopant in optimizing electron rich/poor and poor/poor π-π interactions in such systems. Overall, this structure property analysis sheds light on the molecular level interactions that govern the mechanism by which chirality transfer occurs from dopant to LC host, which is one of the biggest unknowns in the field.

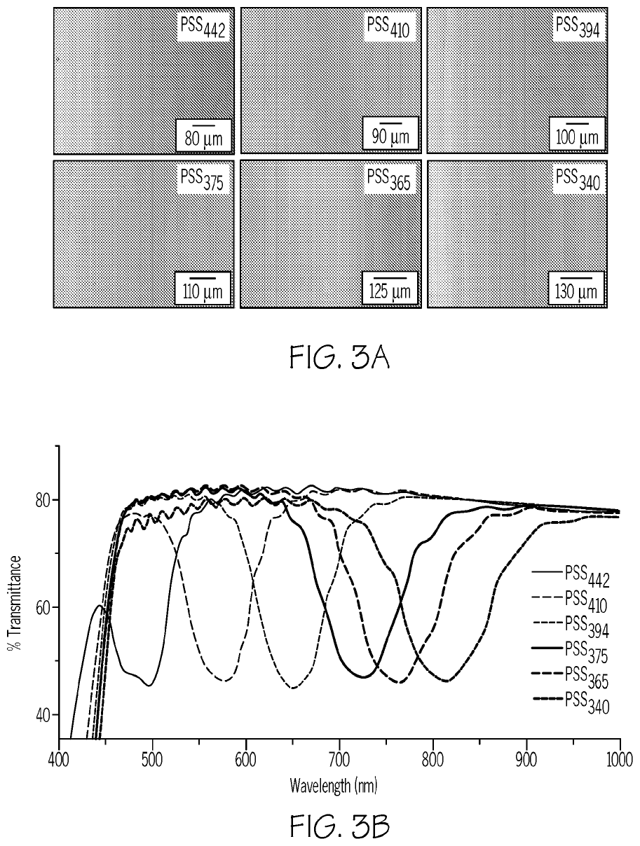

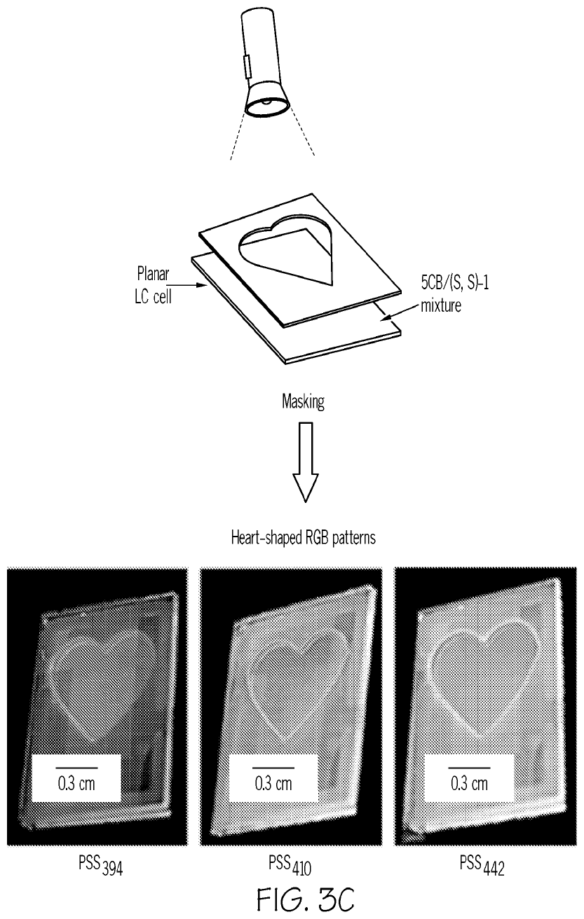

To demonstrate the ability of the dopants to kinetically trap the helical assembly at a particular pitch based on the irradiation wavelength (i.e., PSS), samples of the best performing (i.e., highest β and Δβ, see Table 1) dopants (R, R)-1 and (S, S)-1 (FIG. 3A) in 5CB were irradiated with 340, 365, 375, 394, 410 and 442 nm light until the appropriate PSS was reached (i.e., when the helical pitch no longer changed with continued irradiation).

In general, the P length becomes longer when using shorter irradiation wavelengths. Importantly, the thermal stability of the hydrazone switch allowed Applicant to lock in the different pitches of the LC as a function of their different wavelength dependent PSSs for extended periods of time. Applicant also demonstrated that different pitches can be obtained and locked it as a function of the irradiation time at a given wavelength (e.g., 442 nm (vide-infra)). It is worth bearing in mind that the LC system is a supramolecular self-assembled helical superstructure, and Applicant is kinetically (i.e., out-of-equilibrium) trapping these different self-assemblies as a function of irradiation wavelength or time.

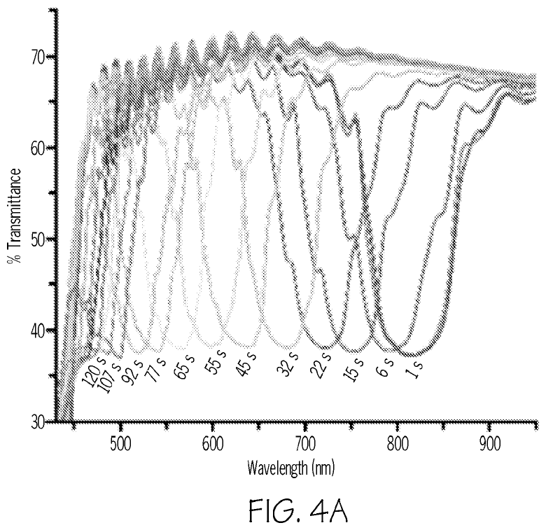

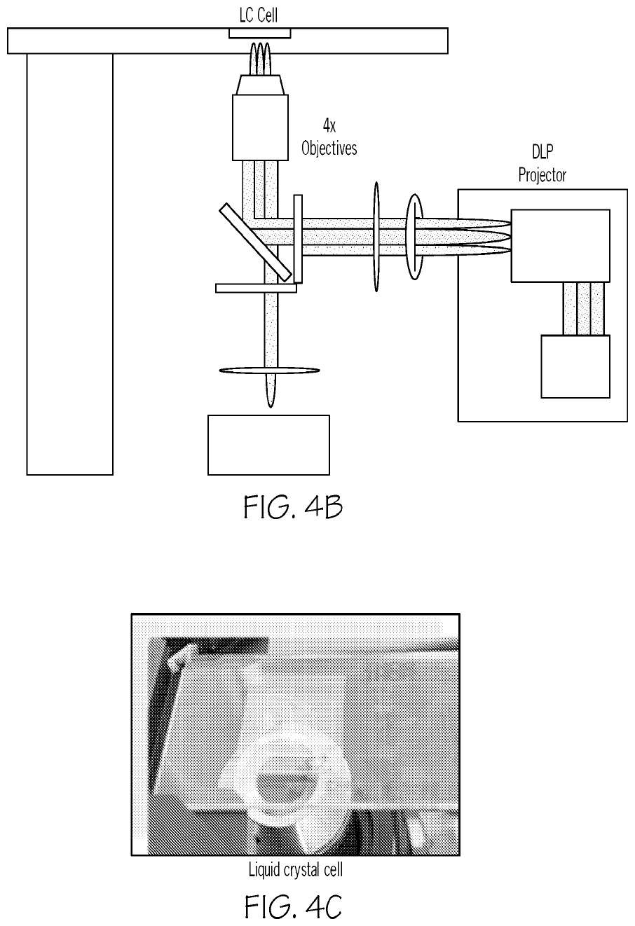

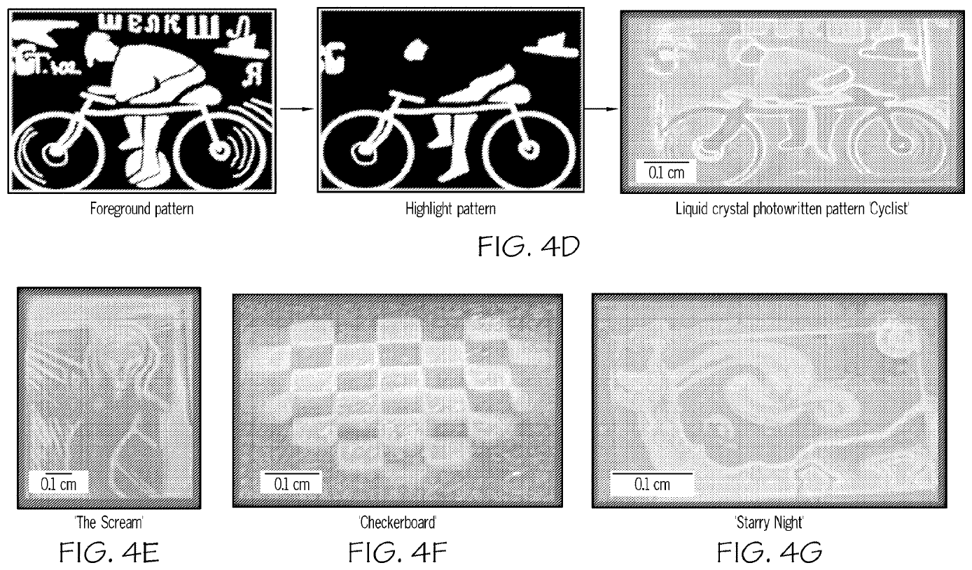

To take the advantage of kinetic trapping property and the relatively large β and Δβ of the dopants, Applicant made adaptive reflective films, by loading doped 5CB into planarly aligned LC cells (5 μm cell thickness). The transmission data obtained from these films, which were measured as a function of irradiation wavelength (FIG. 3B for (S, S)-1) or irradiation times (wavelength fixed at 442 nm) (FIG. 4A for (S, S)-1)) show optimal control over the reflection color from the visible to the NIR region (e.g., from 485 to 812 nm when scanning the wavelength from 442 to 340 nm, respectively).

Importantly the thermal stability of the hydrazones allowed Applicant to lock-in all these states and associated reflected colors for extended periods of time (weeks). To showcase the abilities of the new dopant, Applicant engineered a system such that it can reflect the primary colors, RGB, at PSS₃₉₄, PSS₄₁₀ and PSS₄₄₂, respectively, and then used masks to draw RGB colored heart-shaped patterns using the LC films (FIG. 3C). As for dopants 2-7, their β and Δβ values are moderate enough to enable the fine adjustment of light reflectance even in the visible region.

Considering that Applicant has a unique way of controlling over the reflected colors and their stability, Applicant decided to use the LC films as canvases on which Applicant can draw multi-color images, which can be erased on demand or locked-in for extended periods of time. Using a DLP microscope projection (FIGS. 4B-4C), Applicant patterned multi-color images by controlling illumination times (FIGS. 4D-4G) of patterned blue LED light centered at 450 nm. For dual-color images, the desired images were deconstructed using GNU Image Manipulation Program (GIMP 2.10.30) into a processed image for patterning a blue foreground with a green background using 16 seconds illumination time. Following this procedure, a dual-color image of Edvard Munch's “The Scream” was printed onto the liquid crystal canvas (FIG. 4E). Inclusion of more colors was readily achieved by deconstructing the image into two patterns for a “foreground” and “highlight” by illuminating the “foreground” pattern for 4 seconds, illuminating the “highlight” pattern for 4-12 seconds, and leaving an unpatterned background for the third color. This procedure was used to generate unprecedented CLC multi-color images (FIG. 4F), including Van Gogh's “Starry Night” (FIG. 4G) and Natalia Goncharova's “Cyclist” (FIG. 4D).

### Example 1.3. Conclusions

In this Example, Applicant demonstrates the first use of triptycene as the chiral element of a photo switchable dopant. The fact that this scaffold results in a large β value drastically expands the chiral unit toolbox available for practitioners, which has so far mainly focused on binaphthyl derivatives. The combination of triptycene with the large geometrical change that hydrazones undergo upon photoisomerization results in a relatively large Δβ value. The structure property analysis yielded much needed insights about the molecular level mechanisms that control the chirality transfer between the dopant and host LC (i.e., β and Δβ values). This information will be of outmost importance to practitioners who are interested in designing efficient chiral dopants.

Finally, the Δβ of dopant 1 allowed for the modulation of the LC reflected light from the visible, a first for hydrazones, to the NIR range (450-800 nm, when changing irradiation time). On the other hand, the bi-stability of the hydrazone enabled the long-term locking-in of the reflected color as a function of wavelength, yielding a surface that can be patterned to reflect the primary RGB colors. The same property also allowed Applicant to use the irradiation time of visible light (442 nm) to lock-in different colors, also a new concept, which when combined with a simple DLP microscope projection setup produces multi-color pictures, including Natalia Goncharova's “Cyclist” on the LC canvas, which is also a new development. These results showcase the unique properties that hydrazones, with their bi-stability and large geometrical changes, offer, which when combined with the large β value of triptycene, yields unprecedented control over LC color reflection.

### Example 1.4. Methods

All reagents and starting materials were obtained from commercial sources and used without further purification. All reactions were done under normal atmosphere unless otherwise noted. Compounds were purified by column chromatography using silica gel (SiliCycle®, 60 Å, 230-400 mesh) as the stationary phase and eluting solvents are reported as ratios unless otherwise noted. Racemic 2,6-diaminotriptycene was resolved into enantiomers (R, R)-2 and (S, S)-2 by chiral-phase HPLC using a CHIRALCEL® OD-H column (MeOH+0.1% Et₂NH) with enantiomeric excess (% ee) of 98.3% (yield: 69.4%) and 98.9% (yield: 76.0%), respectively. Recrystallizations were performed with HPLC grade solvents. Deuterated solvents were obtained from Cambridge Isotope Labs and used without further purification. ¹H and ¹³C NMR spectra were recorded on 500 or 600 MHz instruments with working frequencies of 500.13 and 600.13 MHz for ¹H nuclei and 125.8 or 150.9 MHz for ¹³C nuclei, respectively. Chemical shifts are quoted in ppm relative to tetramethylsilane (TMS), using the residual solvent peak as the reference standard. ESI mass spectra were obtained on a Shimadzu LCMS-8030 mass spectrometer. UV-Vis and transmittance spectra were recorded on a Shimadzu UV-1800 UV-Vis spectrophotometer. Irradiation experiments were conducted with a stand-alone xenon arc lamp system (Model: LB-LS/30, Sutter Instrument Co.), outfitted with a SMART SHUTTER controller (Model: LB10-B/IQ, Sutter Instrument Co.) and a liquid light guide LLG/250. 340 (part number: 340HC10-25), 365 (365HC10-25), 375 (375HC10-25), 394 (part number: 394HC10-25) and 410 (part number: 410FS10-25) nm light filters, purchased from Andover Corporation, were used in the irradiation experiments. Circular Dichroism (CD) spectra were recorded in 1 mm pathlength quartz cuvette using JASCO J-815 CD spectrometer equipped with a Peltier thermostat.

### Example 1.5. Liquid Crystal (LC) Systems

The LC nematic host 5CB was purchased from TCI. Doped LC samples were prepared using accurately weighed samples (using an analytical balance) of the chiral dopant and 5CB nematic host. The cholesteric liquid crystal (CLC) mixtures were made by dissolving both components in a few drops of spectroscopic grade dichloromethane, followed by the overnight evaporation of the solvent under high vacuum to ensure that no traces of the solvent is left in the CLC mixtures. The textures of the LC samples were observed using an Olympus BX53 polarized optical microscope, and the photomicrographs were captured using an INSTEC MITO2-MC camera. The planar LC cells (LC3-5.0) were purchased from INSTEC, and the KCRK07 (θ=0.0183) wedge cells were purchased from EHC Japan. For photo-patterning experiments, a heart-shaped black flexible plastic mask was placed on top of the planar LC cell and irradiated with specific wavelengths of light. For the DLP experiments, LC cells were placed on top of a microscope slide and mounted on the DLP microscope. To expand the area of the projection and to fill the cell, a 50 mm achromatic doublet was used as a collimating lens as opposed to the previously described 100 mm lens. The projected image was focused and aligned using weak red light (0.022 W cm⁻², 5% PWM), while patterning was performed with strong blue light (0.475 W cm⁻², 100% PWM), with color variation achieved via varying exposure time as opposed to intensity.

In sum, Applicant has developed a new type of photo-switchable chiral dopant that can be used in tuning a reflected color of a liquid crystal film. Advantageously, the hydrazone switch used in the dopant is bistable, allowing not only access to primary color reflection (i.e., red, green and blue) but also indefinitely locking-in of these colors and allowing for the use of such systems in reconfigurable display applications that do not require electronics.

Applicant has shown that many different color reflections can be accessed using the new dopant. Additionally, as further illustrated in FIGS. 3A-3C and 4A-4G, Applicant has proof of principle demonstration of potential applications of this technology.

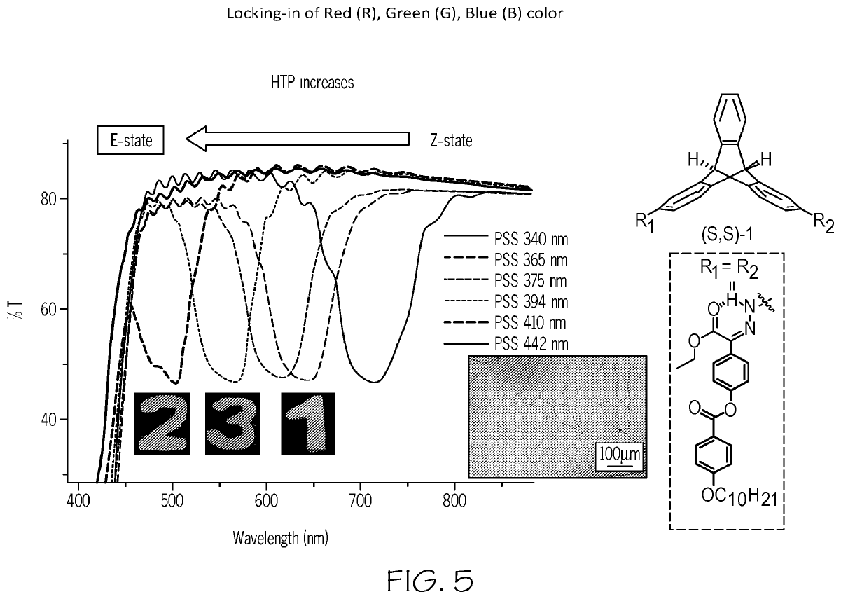

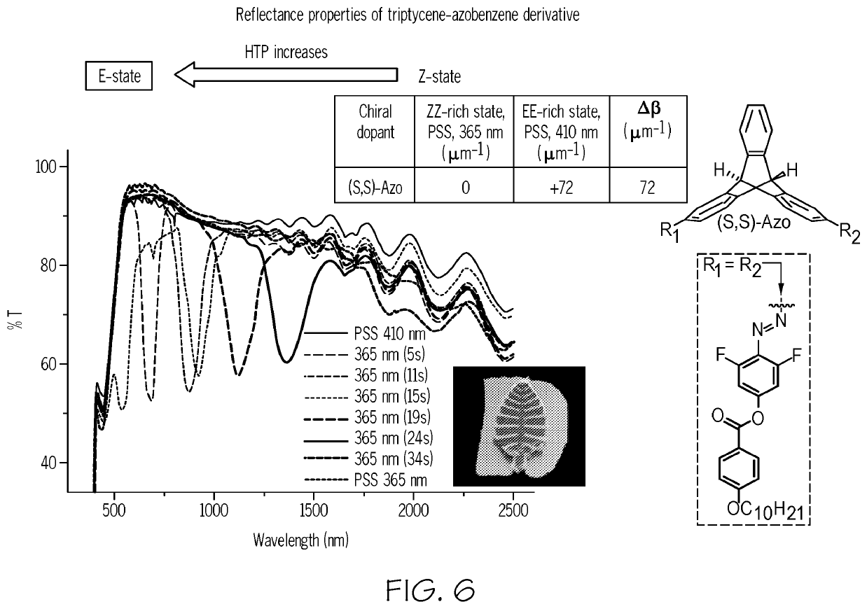

FIGS. 5-6 illustrate additional proof of principle demonstrations. In particular, FIG. 6 provides preliminary results using an (S,S)-Azo derivative. The results demonstrate that the derivative is also an acceptable dopant for substrates. The dopant (0.2 mol % doped in 5CB) in its PSS₄₁₀ (mainly the EE isomer) shows a β value of +72 μm⁻¹, which upon irradiation with 365 nm light yields the ZZ rich dopant that yields a β value of 0 μm⁻¹, resulting in the largest Δβ value (+72 μm⁻¹) obtained so far in the triptycene-based dopants. The thermal stability of the dopant is less than those of the hydrazone-based systems with a half-life of 28 days, which is still viable for long term applications. The ability of the dopant to control reflected light was studied by varying the irradiation wavelength (PSS-dependent reflection) and by controlling the irradiation time at 365 nm. The latter allowed Applicant to control the reflection wavelength from ca. 500 nm to 2500 nm, thus covering almost all the visible range to the NIR. This level of control allowed Applicant to draw multi-color images, such as the Dartmouth logo as shown in FIG. 6.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A chiral compound, wherein the chiral compound is selected from the group consisting of: 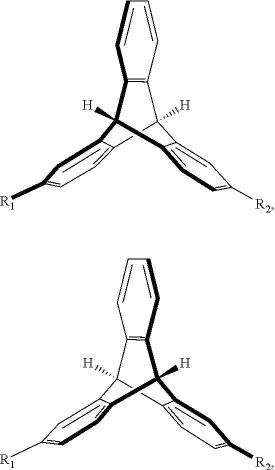
or derivatives thereof,
wherein at least one of R₁ and R₂ comprises a photo-switchable unit, and
wherein R₁ and R₂ are each independently selected from the group consisting of alkyl groups, alkene groups, alkenyl groups, alkoxy groups, hydroxy groups, aryl groups, ketone groups, aldehyde groups, amine groups, amide groups, imine groups, carboxyl groups, carboxylic acid groups, ester groups, thiol groups, sulfoxide groups, alcohol groups, alkyne groups, azide groups, hydrazide groups, hydrazone groups, azo groups, cyano groups, nitro groups, halogens, a methyl group, a polymer, a substrate component, a photo-switchable unit, or combinations thereof.

2. The compound of claim 1, wherein R₁ and R₂ each comprise a photo-switchable unit selected from the group consisting of a hydrazone unit, an azobenzene unit, or combinations thereof.

3-4. (canceled)

5. The compound of claim 1, wherein the photo-switchable unit comprises a structure selected from the group consisting of: 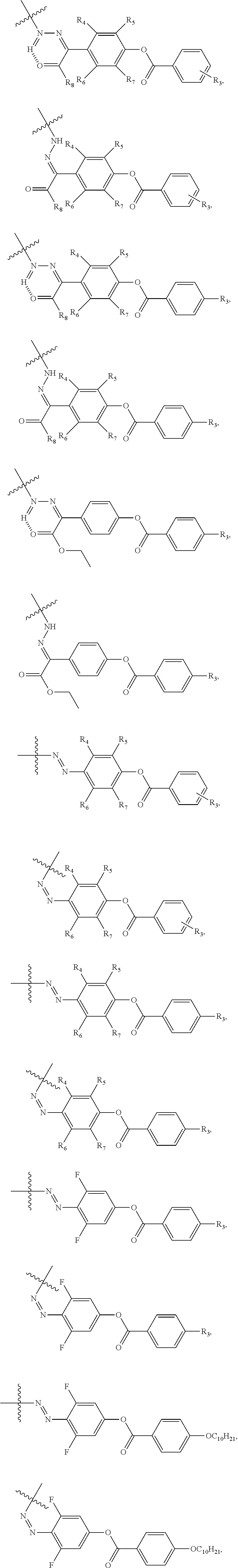
derivatives thereof, or combinations thereof,
wherein R₃, R₄, R₅, R₆, R₇ and R₈ are each independently selected from the group consisting of H, alkyl groups, alkene groups, alkenyl groups, alkoxy groups, hydroxy groups, aryl groups, ketone groups, aldehyde groups, amine groups, amide groups, carboxyl groups, carboxylic acid groups, ester groups, thiol groups, sulfoxide groups, alcohol groups, alkyne groups, azide groups, halogens, CN, OC₁₀H₂₁, —OCH₃, NO₂, F, N(CH₃)₂, a methyl group, a polymer, a substrate component, a photo-switchable unit, or combinations thereof.

6. The compound of claim 1, wherein each of R₁ and R₂ comprises the following structure: 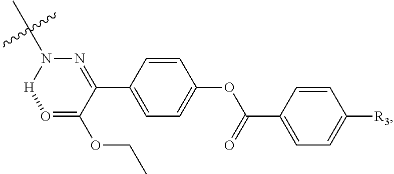
wherein R₃ is OC₁₀H₂₁.

7. The compound of claim 1, wherein each of R₁ and R₂ comprises the following structure: 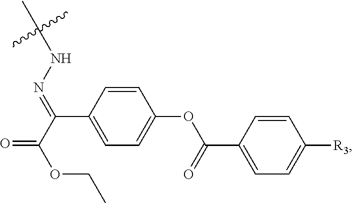
wherein R₃ is OC₁₀H₂₁.

8. A substrate comprising one or more regions, wherein the one or more regions are associated with a chiral compound selected from the group consisting of: 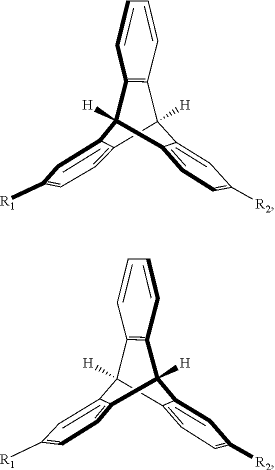
or derivatives thereof,
wherein at least one of R₁ and R₂ comprises a photo-switchable unit, and
wherein R₁ and R₂ are each independently selected from the group consisting of alkyl groups, alkene groups, alkenyl groups, alkoxy groups, hydroxy groups, aryl groups, ketone groups, aldehyde groups, amine groups, amide groups, imine groups, carboxyl groups, carboxylic acid groups, ester groups, thiol groups, sulfoxide groups, alcohol groups, alkyne groups, azide groups, hydrazide groups, hydrazone groups, azo groups, cyano groups, nitro groups, halogens, a methyl group, a polymer, a substrate component, a photo-switchable unit, or combinations thereof.

9. The substrate of claim 8, wherein R₁ and R₂ each comprise a photo-switchable unit selected from the group consisting of a hydrazone unit, an azobenzene unit, or combinations thereof.

10-11. (canceled)

12. The substrate of claim 8, wherein the photo-switchable unit comprises a structure selected from the group consisting of: 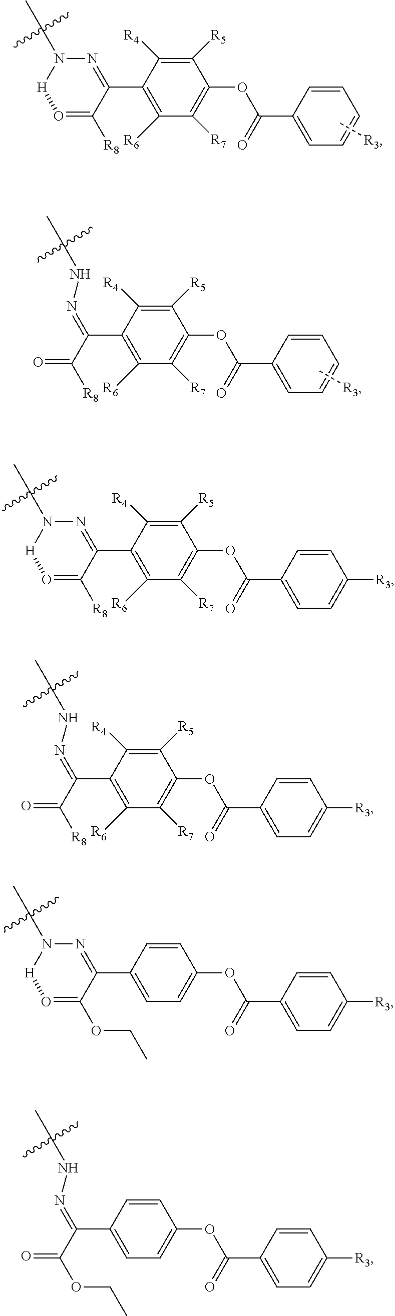 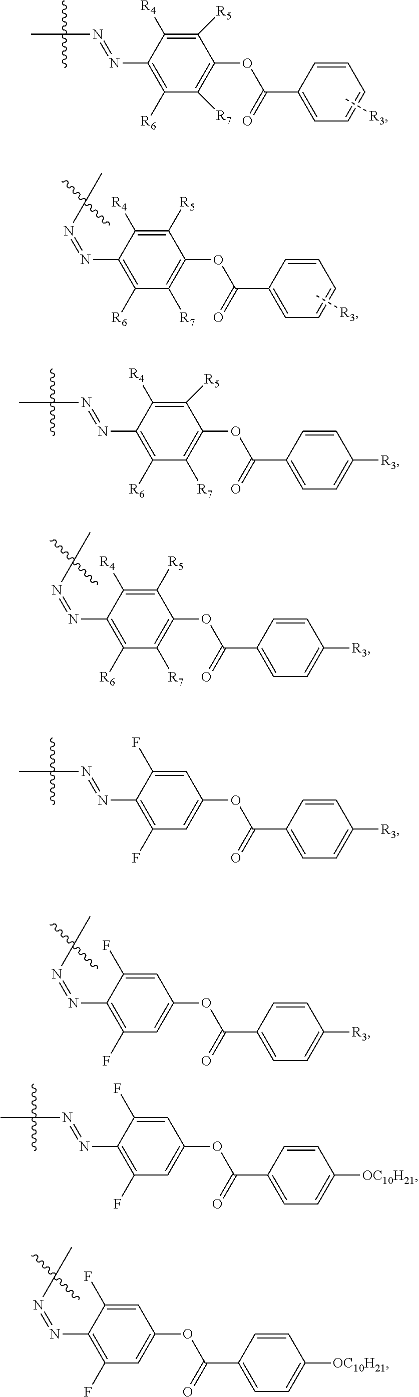
derivatives thereof, or combinations thereof,
wherein R₃, R₄, R₅, R₆, R₇ and R₈ are each independently selected from the group consisting of H, alkyl groups, alkene groups, alkenyl groups, alkoxy groups, hydroxy groups, aryl groups, ketone groups, aldehyde groups, amine groups, amide groups, carboxyl groups, carboxylic acid groups, ester groups, thiol groups, sulfoxide groups, alcohol groups, alkyne groups, azide groups, halogens, CN, OC₁₀H₂₁, —OCH₃, NO₂, F, N(CH₃)₂, a methyl group, a polymer, a substrate component, a photo-switchable unit, or combinations thereof.

13. The substrate of claim 8, wherein each of R₁ and R₂ comprises the following structure: 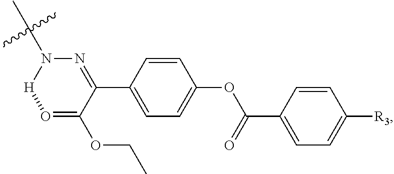
wherein R₃ is OC₁₀H₂₁.

14. The substrate of claim 8, wherein each of R₁ and R₂ comprises the following structure: 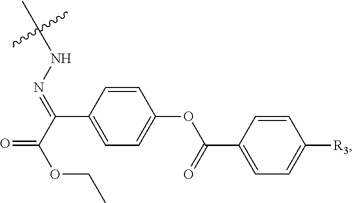
wherein R₃ is OC₁₀H₂₁.

15. The substrate of claim 8, wherein the substrate is selected from the group consisting of a film, a liquid crystal film, a display panel film, a mask surface, a polymer, a polydimethylsiloxane film, a display screen, a paper, or combinations thereof.

16. The substrate of claim 8, wherein the substrate comprises a liquid crystal film.

17. The substrate of claim 8, wherein the substrate comprises a plurality of different regions, wherein the different regions of the substrate comprise different or the same compounds of claim 8.

18-19. (canceled)

20. A method of changing a color of one or more regions of a substrate, said method comprising:
applying light to the one or more regions of the substrate,
wherein the one or more regions of the substrate comprises a chiral compound selected from the group consisting of 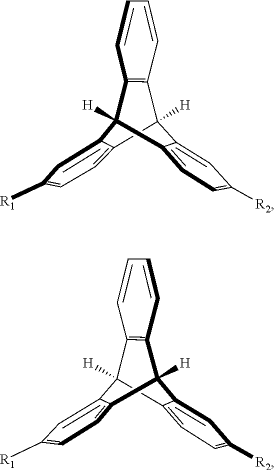
or derivatives thereof,
wherein at least one of R₁ and R₂ comprises a photo-switchable unit, and
wherein R₁ and R₂ are each independently selected from the group consisting of alkyl groups, alkene groups, alkenyl groups, alkoxy groups, hydroxy groups, aryl groups, ketone groups, aldehyde groups, amine groups, amide groups, imine groups, carboxyl groups, carboxylic acid groups, ester groups, thiol groups, sulfoxide groups, alcohol groups, alkyne groups, azide groups, hydrazide groups, hydrazone groups, azo groups, cyano groups, nitro groups, halogens, a methyl group, a polymer, a substrate component, a photo-switchable unit, or combinations thereof.

21. The method of claim 20, wherein R₁ and R₂ each comprise a photo-switchable unit selected from the group consisting of a hydrazone unit, an azobenzene unit, or combinations thereof.

22-23. (canceled)

24. The method of claim 20, wherein the photo-switchable unit comprises a structure selected from the group consisting of: 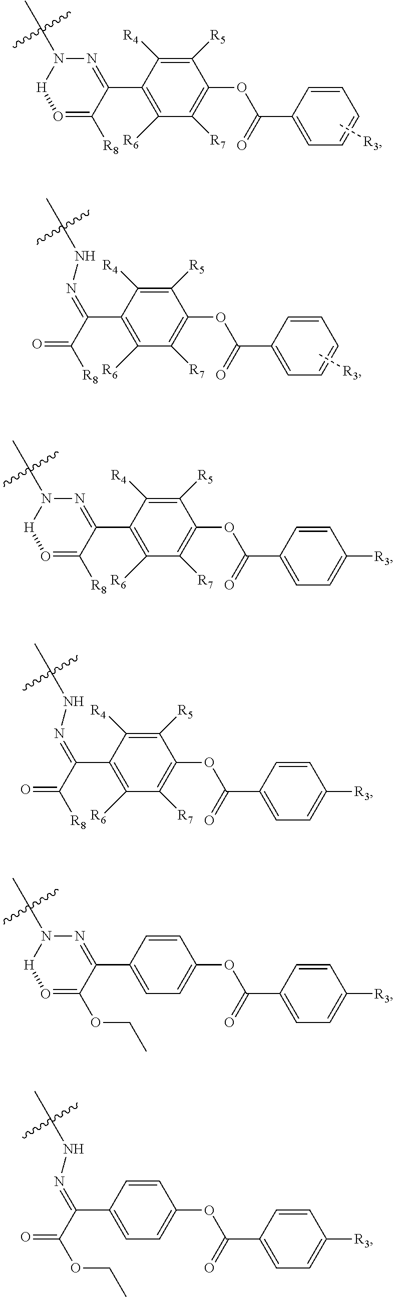 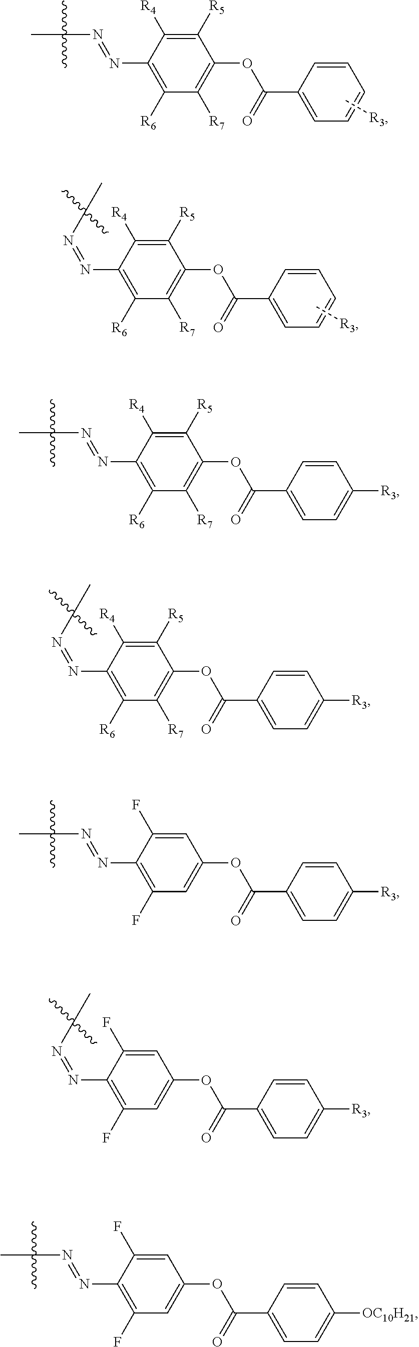 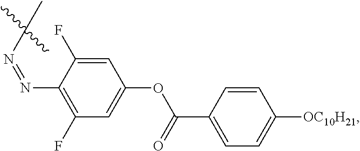
derivatives thereof, or combinations thereof,
wherein R₃, R₄, R₅, R₆, R₇ and R₈ are each independently selected from the group consisting of H, alkyl groups, alkene groups, alkenyl groups, alkoxy groups, hydroxy groups, aryl groups, ketone groups, aldehyde groups, amine groups, amide groups, carboxyl groups, carboxylic acid groups, ester groups, thiol groups, sulfoxide groups, alcohol groups, alkyne groups, azide groups, halogens, CN, OC₁₀H₂₁, —OCH₃, NO₂, F, N(CH₃)₂, a methyl group, a polymer, a substrate component, a photo-switchable unit, or combinations thereof.

25. The method of claim 20, wherein each of R₁ and R₂ comprises the following structure: 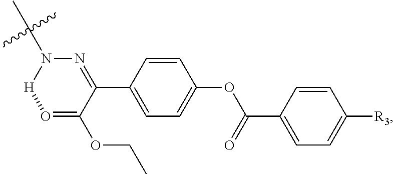
wherein R₃ is OC₁₀H₂₁.

26. The method of claim 20, wherein each of R₁ and R₂ comprises the following structure: 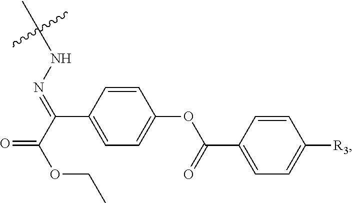
wherein R₃ is OC₁₀H₂₁.

27. The method of claim 20, wherein the light comprises a wavelength ranging from about 300 nm to about 900 nm.

28. The method of claim 20, wherein light is applied to one or more regions of a substrate for a time period ranging from about 1 second to about 120 seconds.

29. The method of claim 20, wherein the application of the light forms a color pattern on the substrate.

30. The method of claim 20, wherein the same wavelength of light is applied to different regions of the substrate to result in the same color change in the different regions of the substrate, different wavelengths of light are applied to different regions of the substrate to result in different color changes in the different regions of the substrate, different wavelengths of light are applied to the same regions of the substrate to result in different color changes in the same regions of the substrate, the light is applied to the same region of the substrate for the same periods of time to result in the same color change in the same region of the substrate, the light is applied to the same region of the substrate for different periods of time to result in different color changes in the same regions of the substrate, the light is applied to different regions of the substrate for the same periods of time to result in the same color change in the different regions of the substrate, the light is applied to different regions of the substrate for different periods of time to result in different color changes in the different regions of the substrate, or combinations thereof.

31-35. (canceled)

37. The method of claim 20, wherein the applying of the light to the one or more regions of the substrate comprises:
applying a first light to the one or more regions of the substrate, wherein the first light changes the color of the one or more regions of the substrate to a first color; and
applying a second light to the one or more regions of the substrate, wherein the application of the second light changes the color of the one or more regions of the substrate to a second color.

38. The method of claim 37, wherein the first light and the second light have different or the same wavelengths.

39. (canceled)

40. The method of claim 37, wherein the first light and the second light are applied to the one or more regions of the substrate for different or the same periods of time.

41. (canceled)

42. The method of claim 37, wherein the first light and the second light are applied to the same or different regions of the substrate.

43. (canceled)

44. The method of claim 20, wherein the applying of the light to the one or more regions of the substrate comprises:
applying a first light to the one or more regions of the substrate, wherein the first light changes the color of the one or more regions of the substrate; and
applying a second light to the one or more regions of the substrate, wherein the application of the second light occurs after the application of the first light to the substrate, and wherein the application of the second light reverses the change in the color of the one or more regions of the substrate.

45. The method of claim 44, wherein the first light comprises light at a first wavelength, wherein the second light comprises light at a second wavelength, wherein the first wavelength is about 442 nm, and wherein the second wavelength is about 340 nm.

46. (canceled)

47. The method of claim 20, wherein the change in color comprises a change in color to red, green, blue, purple, pink, orange, yellow, or combinations thereof.

48. (canceled)

49. The method of claim 20, wherein the change in color is in the form of a pattern on the substrate.

50. The method of claim 20, wherein the substrate is selected from the group consisting of a film, a liquid crystal film, a display panel film, a mask surface, a polymer, a polydimethylsiloxane film, a display screen, a paper, or combinations thereof.

51. The method of claim 20, wherein the substrate comprises a liquid crystal film.

52. The method of claim 20, wherein the substrate comprises a plurality of different regions, wherein each of the plurality of different regions are associated with the one or more compounds of claim 1, wherein the different regions of the substrate comprise different compounds of claim 1, or wherein the different regions of the substrate comprise the same compounds of claim 1.

53-54. (canceled)
